(19) United States
(12) Patent Application Publication
(10) Pub. No.: US 2021/0169399 A1
(43) Pub. Date: Jun. 10, 2021

(54) Device and Method for Detecting Uterine Activity

(71) Applicants: Himanshu Nandlal Shah, Prospect, KY (US); Riya Himanshu Shah, Prospect, KY (US)

(72) Inventors: Himanshu Nandlal Shah, Prospect, KY (US); Riya Himanshu Shah, Prospect, KY (US)

(21) Appl. No.: 16/707,821

(22) Filed: Dec. 9, 2019

(51) Int. Cl. A61B 5/00; A61B 5/11; G01L 1/22; G01S 19/01; G16H 40/63

(52) U.S. Cl. CPC A61B 5/4356; A61B 5/1112; G01L 1/2293; G01S 19/01; A61B 2562/0261; A61B 5/6831; G16H 40/63; G01L 1/2206; A61B 5/0022

(57) ABSTRACT

A device is provided having a sensor for detecting uterine activity of a pregnant user. The device may be positioned abutting the user's abdomen, and includes a housing defining an inner cavity with a circuit board positioned therein. The circuit board includes at least one slot defining a cantilever portion thereof having an attached end and a free end, and the sensor may be positioned proximate the attached end. An actuator may be coupled to the free end of the cantilever portion such that movement of the actuator (e.g., as a result of a contraction) causes the cantilever portion to flex, and the sensor to detect a resulting strain on the cantilever portion.

### FIELD

The present invention relates generally to a sensor device and, more particularly, to a sensor device and method for detecting uterine activity.

### BACKGROUND

Detecting and analyzing uterine activity during pregnancy can yield significant information concerning the condition of the fetus and the advancement of labor. Such monitoring can be especially important during difficult pregnancies which present an increased risk to the health of the fetus. During the second and third trimesters, pregnant women may experience early contractions commonly referred to as Braxton Hicks contractions. Although such contractions do not indicate that labor has begun, it can be difficult for pregnant women to discern the difference between Braxton Hicks contractions and contractions present during first stage labor. As such, it may be desirable to detect such contractions and the frequency at which they occur to determine whether the woman has in fact entered labor

Various devices are generally known for measuring contraction frequency, duration, intensity, and resting tone of the user. These devices may be placed either internally or externally on the patient's abdomen to detect changes in uterine pressure. One example internal device is an intra-uterine pressure catheter. Such catheters may be placed inside the uterus, alongside the fetus, to measure the pressure generated by uterine contractions. However, internal devices are invasive and can be difficult to use. Some known external devices include internal pressure transducers and are placed proximate a pregnant woman's abdomen. Upon impingement by the abdomen during a contraction, an internal pressure of the device rises and is measured. However, it would be desirable to provide a device having an improved sensor for detecting uterine activity.

### SUMMARY

Generally, a device is provided having a sensor for detecting uterine activity of a pregnant user. The device may be positioned externally abutting the user's abdomen, and includes a housing defining an inner cavity with a circuit board positioned therein. The circuit board includes at least one slot defining a cantilever portion thereof having an attached end and a free end with the sensor positioned proximate the attached end. An actuator may be coupled to the free end of the cantilever portion such that movement of the actuator (e.g., as a result of a contraction) causes the cantilever portion to flex and the sensor to detect a resulting strain on the cantilever portion. The strain detected by the sensor or the strain detected over a period of time may thereafter be used to determine, for example, whether a contraction has occurred or whether the user has entered labor.

The circuit board including the cantilever portion and the sensor may include additional circuitry and components for converting, processing, conditioning, and/or communicating a signal representative of the detected strain on the cantilever portion. In some forms, the circuit board includes a signal converter, a controller, and communication circuitry. Such components may be electrically connected via wires, connectors, traces, or the like. For example, the sensor may be electrically connected to a signal converter such that an analog signal representative of strain detected by the sensor may be converted to a digital signal for the controller. The controller may then cause the communication circuitry to communicate the digital signal representative of the detected strain to a computing device, such as a remote computing device or a mobile communication device (which may include a computing device).

A processor of the remote computing device or mobile communication device may be configured to determine whether one or more contractions have occurred based at least in part on a comparison between the signal representative of the detected strain of the cantilever portion and a threshold. Further, either the remote computing device or mobile communication device may determine whether the user has entered the first stage of labor based on, for example, a measured time interval between detected contractions. Communication circuitry of the computing device may be configured to communicate information corresponding to detected contractions to, for example, a mobile communications device. The communication circuitry may also be configured to communicate a notification or alert to a mobile communication device of the user or other person or device upon the computing device determining that the user has entered the first stage of labor, or whether the measured contractions are a “false alarm” (i.e., Braxton Hicks contractions, or other muscle contractions).

Alternatively, the circuit board may include a processor configured to determine whether the user has entered into first stage labor, and the communication circuitry of the board may be configured to communicate information or data corresponding to detected contractions and to communicate a notification or alert to a mobile communication device.

Additionally, the circuit board may include one or more apertures positioned proximate the attached end of the cantilever portion and sized to receive fasteners therethrough for coupling the circuit board to the housing. Alternatively, a portion of the circuit board proximate the attached end of the cantilever portion may be anchored to the housing by other means, such as an adhesive, clips, or screws. This coupling is configured to promote increased stability of the circuit board near the attached end of the cantilever portion as the free end thereof is flexed or otherwise displaced by the actuator causing the cantilever portion to flex.

In some forms, the actuator may be formed as a generally annular button that may be positioned proximate the user's abdomen. The body mass index (BMI) or body type of a user may require an actuator having a correspondingly appropriate size and configuration (e.g., an axial length and/or surface area for contacting the abdomen). As such, the present disclosure likewise provides alternative actuators that may be selectively coupled to the device and interchangeable such that the user may select a desired actuator to be used. Likewise, a kit may be provided including a device without an actuator attached, one or more embodiments of actuators, and a fastener for selectively coupling the actuators to the device.

A method for detecting uterine activity is additionally provided including the steps of providing a sensor device as disclosed above and detecting the strain on the cantilever portion via the sensor. Thereafter, the method may include communicating a signal representative of the detected strain on the cantilever portion to a remote computing device or mobile communication device and determining whether a contraction has occurred based at least in part on a comparison between the detected strain and a threshold. Based at least in part on a measured time interval between detected contractions, the computing device may be configured to determine whether the user has entered the first stage of labor. Optionally, the method may further include the step of communicating a notification signal to a mobile communication device of the user or another person upon determining that labor has begun.

### BRIEF DESCRIPTION OF THE DRAWINGS

FIG. 1 is a front perspective view of an example uterine activity sensor device showing a housing thereof;

FIG. 2 is a rear perspective view showing the actuator;

FIG. 3 is a top plan view of the device shown in FIG. 1;

FIG. 4A is a view of the front side (i.e., the side facing away from a patient user) of the uterine activity sensor device of FIG. 1 with the rear side of the housing removed to show a circuit board disposed in an inner cavity thereof;

FIG. 4B shows the rear side of the housing that was removed in FIG. 4A looking from inside the housing;

FIG. 5 is a perspective view of the front side of the device in FIG. 4A showing the circuit board flipped over and revealing a power source electrically coupled thereto;

FIG. 6A is a front view of the circuit board shown in FIGS. 4A and 5 showing the cantilever portion and a sensor positioned proximate an attached end thereof;

FIG. 6B shows an alternative embodiment of the cantilever portion of the circuit board, with the cantilever portion disposed at an angle;

FIG. 6C shows another alternative embodiment of the cantilever portion of the circuit board;

FIG. 6D shows yet another alternative embodiment of the cantilever portion of the circuit board;

FIG. 7 is a perspective view of a portion of the circuit board showing part of an actuator coupled to the free end of the cantilever portion in a resting state;

FIG. 8 is a perspective view, showing the part of the actuator in FIG. 7 being displaced in an axial direction and deforming the cantilever portion;

FIG. 9 is a perspective view showing a band coupled to the device of FIG. 1;

FIG. 10A is a perspective view showing the device in FIG. 2 with an alternative embodiment of the actuator;

FIG. 10B is a perspective view showing the device in FIG. 2 with another alternative embodiment of the actuator;

FIG. 11 is a block diagram of the uterine activity sensor device shown in FIG. 1;

FIG. 12 is a block diagram showing communication functionality of the uterine activity sensor device of FIG. 1;

FIG. 13 is a flowchart showing an example of a method of using a sensor device for detecting uterine activity; and

FIG. 14 is an example kit including a uterine activity sensor device, interchangeable actuator embodiments, and an example fastener.

Terms of orientation are for convenient reference to the drawings and are not intended to limit the orientation of the sensor device in use.

### DETAILED DESCRIPTION

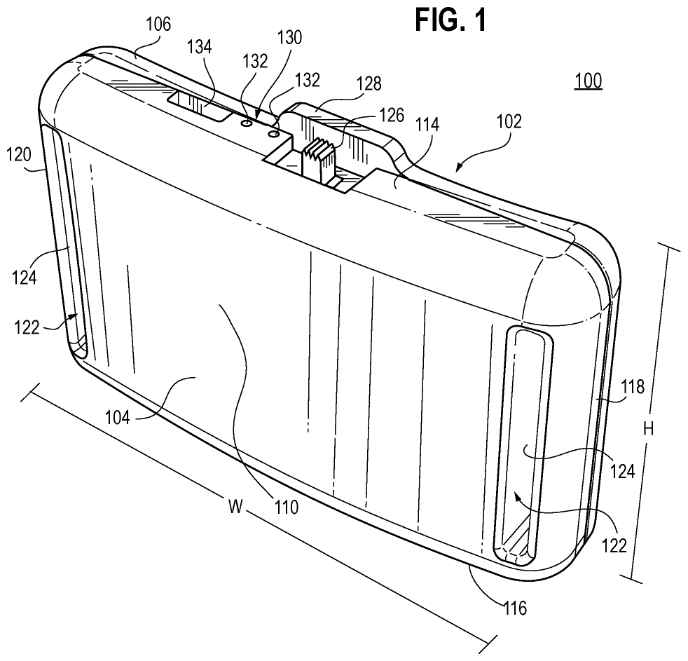

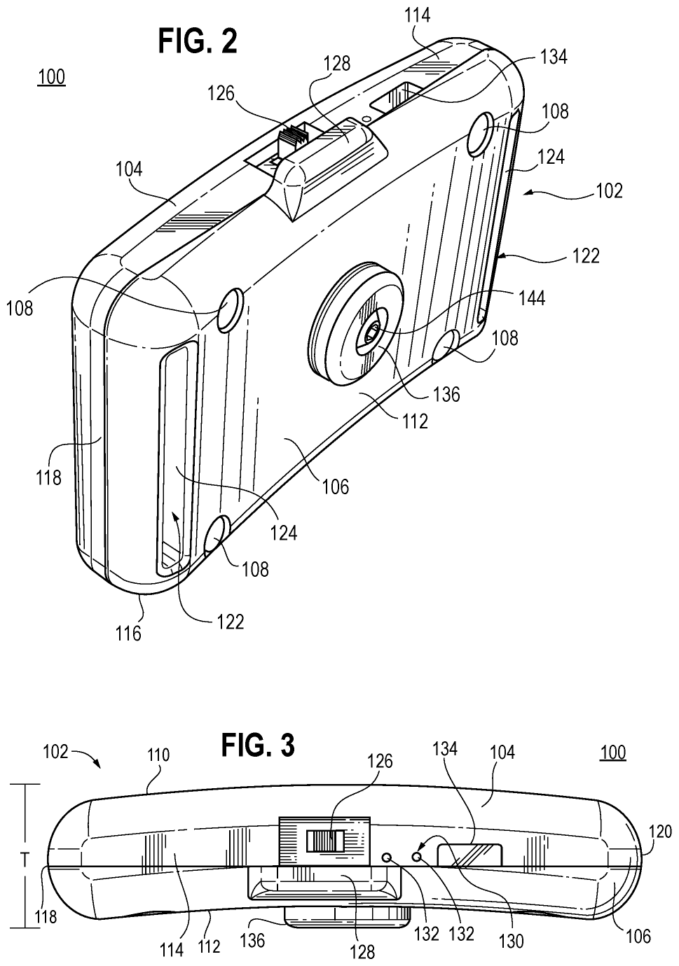

Referring now to the drawings, and more particularly FIGS. 1-3, a sensor device 100 is provided having a housing 102 defining an inner cavity therein. As illustrated, the housing 102 of the device 100 includes a front side 104 and a back side 106 (i.e., the user-facing side) that may be coupled together to form the housing 102. For example, as shown in FIG. 2, the back side 106 of the housing 102 may be coupled to the front side 104 via fasteners 108 positioned in corners of the back side 106. In other forms, the front and back sides 104, 106 may be snap-fit together.

Once assembled, the housing 102 includes a front portion 110, a rear portion 112, an upper portion 114, a lower portion 116, and two side portions 118, 120. The housing 102 and portions 110, 112, 114, 116, 118, 120 thereof preferably include rounded or filleted edges such that the device 100 may be comfortably handled and worn by a user. In addition, the housing 102 is shown having a generally arcuate configuration with the front side 104 having a generally convex shape and the back side 106 having a generally concave shape such that it may generally conform to the abdomen of the user for further improved comfort and effective actuation of an actuator 136 caused by uterine activity. In some forms, the housing 102 may be about 3 inches wide (width W), 2 inches tall (height H), and 0.7 inches thick (thickness T). These example dimensions are provided herein for the purpose of describing the device 100, however, the housing 102 may be formed in a variety of different sizes and shapes.

As illustrated, the housing 102 may include openings 122 in the form of slots 124 extending through the front and back sides 104, 106 for receiving, for example, a belt or strap for securing the device 100 to the abdomen of the user. The slots 124 are shown adjacent the portions 118, 120 of the housing, but may alternatively be positioned in other locations. In other embodiments, the device 100 may include openings 122 having different shapes or configurations for receiving belts or straps of different sizes and shapes.

The device 100 may include one or more switches or controls, such as switch 126 for turning the device 100 on and off. In order to inhibit accidental actuation of the switch 126, a switch guard 128 may be positioned on the back side 106 of the housing 102 such that the abdomen of the user does not inadvertently or unintentionally contact the switch 126 while the device 100 is being worn. As shown, the switch guard 128 is formed integral with the back side 106 of the housing, but in alternative forms may be a separate component coupled thereto. Additionally, one or more indicators 130 such as light emitting diodes (LEDs) 132 may be positioned on the upper portion 114 of the housing 102 and electrically connected to circuitry of the device 100 to indicate an operating status thereof. For example, illumination of the LEDs 132 may indicate to a user that the device 100 is on or off, whether a battery is low, or in other forms may indicate whether the device 100 is successfully paired with a separate device (e.g., a mobile communication device of the user). A charging port 134 may likewise be positioned on the upper portion 114 of the housing 102 and electrically connected to a power source 160 (shown in FIG. 5) of the device 100 to facilitate recharging. Of course, the switch 126, switch guard 128, LEDs 132, and charging port 134 may be positioned at any suitable location on the housing 102 and need not necessarily be placed on the upper portion 114 as shown.

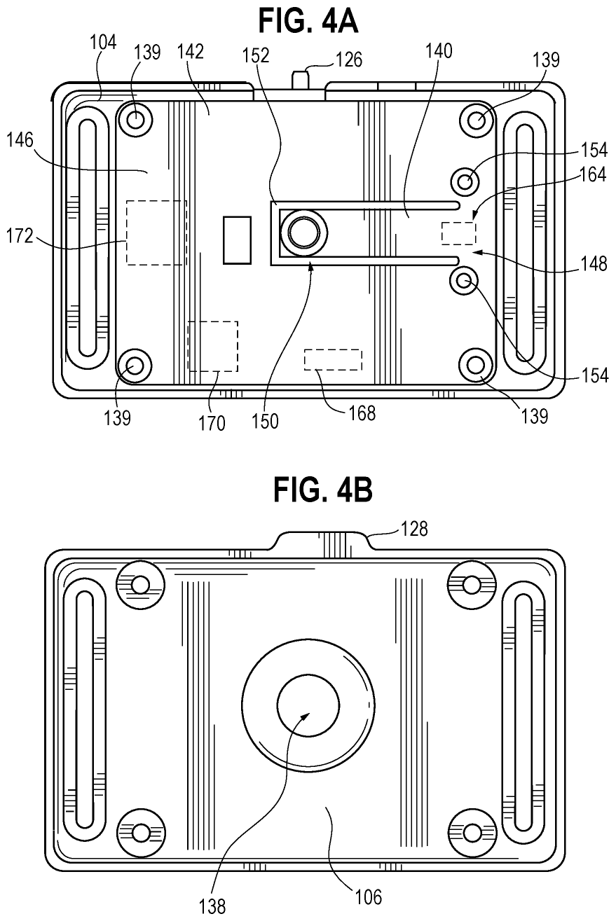

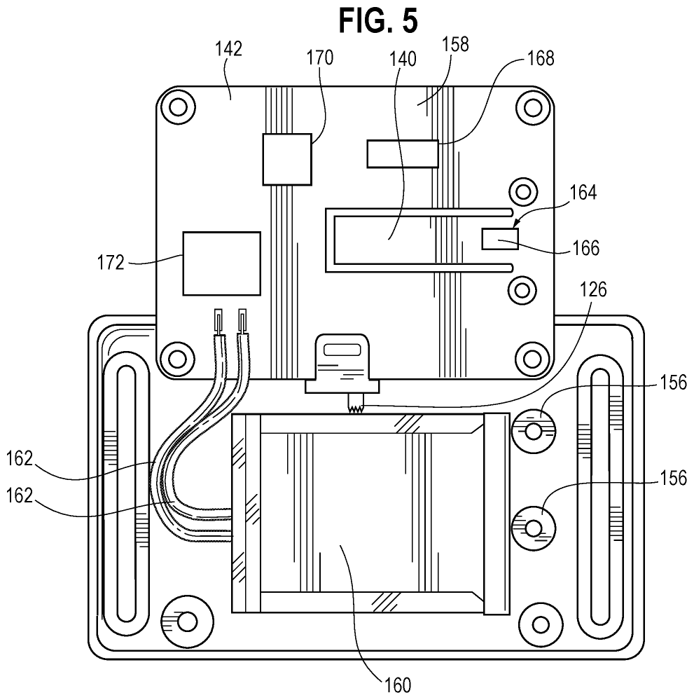

As shown in FIG. 2, the actuator 136 may extend through an aperture 138 (shown in FIGS. 4B and 14) in the back side 106 of the housing 102 and may be coupled to a cantilever portion 140 of a circuit board 142 (shown in FIG. 4A) disposed in the inner cavity. In the illustrated form, the actuator 136 may be coupled to the cantilever portion 140 via a fastener 144 such as a bolt, nut, screw, nail, or the like. Alternatively, the actuator 136 may be coupled to the free end using an adhesive or other fastening means.

As illustrated in FIG. 4A, the back side 106 of the housing 102 has been separated from the front side 104 to show the inner cavity thereof and a first side 146 of the circuit board 142 disposed therein. The circuit board 142 includes apertures 139 for receiving fasteners 108 for attaching the front side 104 to the back side 106 of the device 100. The cantilever portion 140 of the circuit board 142 is illustrated including an attached end 148 and a free end 150. By one approach, the attached end 148 is integral with the circuit board 142 and the free end 150 may include a bore 137 (FIGS. 7 and 8) for receiving the fastener 144 (FIG. 14) to couple the free end 150 to the actuator 136 as described above. In such forms, the free end 150 or a portion thereof is generally aligned with the aperture 138. For example, the threaded fastener 144 may be advanced through an opening of the actuator 136, advanced through the aperture 138, and secured to the bore 137 on the free end 150 of the cantilever portion 140. In some forms, the bore 137 may be threaded for accepting a corresponding threaded fastener. The bore 137 may be a separate structure attached to the free end 150 of the cantilever portion as shown in FIGS. 7 and 8 or may alternatively be formed as a threaded aperture through cantilever portion 140. In still other forms, a fastener such as a threaded fastener may extend from the free end 150 of the cantilever portion 140 and the actuator 136 may include a threaded aperture therethrough such that the actuator may be coupled to the free end 150 by a threaded fastener.

In a preferred embodiment, the cantilever portion 140 is defined by a three-sided slot, such as slot 152, formed in the circuit board 142 so the cantilever portion 140 may bend or flex upon application of force to the free end 150 thereof. As illustrated, the cantilever portion 140 is of a generally rectangular shape, however, the cantilever portion 140 may also have other shapes as described in further detail below. The circuit board 142 also may include one or more apertures, such as apertures 154 positioned proximate the attached end 148 of the cantilever portion 140 that receive fasteners therethrough for coupling, or anchoring, the circuit board 142 to the housing 102 (e.g., through threaded apertures 156 shown in FIG. 5). So configured, the circuit board 142 is anchored at the attached end 148 of the cantilever portion 140 using fasteners (not shown) to promote increased stability of the attached end 148 while allowing the free end 150 to be flexed by the actuator 136. Alternatively, a portion of the circuit board 142 proximate the attached end 148 of the cantilever portion 140 may be anchored to the housing by other means, such as, for example, an adhesive, clips, or screws.

In FIG. 5, the circuit board 142 has been flipped over such that a second side 158 thereof is visible, and to additionally show the power source 160 for the device 100 positioned in the inner cavity and electrically coupled to the circuit board 142 via wires 162. In the preferred form, the second side 158 of the circuit board 142 includes a sensor 164 for detecting the resulting strain on the cantilever portion 140 when the free end 150 thereof is displaced by the actuator 136. The sensor 164 may be printed directly on the circuit board 142 or may alternatively be affixed thereto (e.g., via soldering). In one embodiment, the sensor 164 is configured for detecting strain near the attached end 148 of the cantilever portion 140 where stress concentrations will be the highest. In other embodiments, sensor 164 may be placed closer toward the free end 150 to achieve a desired sensitivity of the sensor 164 to flexing of the free end 150 of the cantilever portion 140. In still other embodiments, multiple sensors 164 may be positioned on or proximate to cantilever portion 140 for detecting a strain on the cantilever portion 140.

In a preferred embodiment, the sensor 164 may comprise a strain gauge 166. For example, the strain gauge 166 may be a foil strain gauge that is affixed to and moves with the cantilever portion 140 of the circuit board 142 such that the resistance across the foil strain gauge changes as the cantilever portion 140 is flexed. In other embodiments, the strain gauge 166 may comprise a silicon strain gauge bonded to the circuit board 142 proximate the attached end 148, or a polymer printed onto cantilevered portion 140 and/or another portion of circuit board 142, to measure strain in a similar manner. Various characteristics and parameters of the strain gauge 166 may be varied such as the resistance and size thereof. In some forms, the strain gauge 166 may include a resistance between about 120 to about 350 ohms. So configured, and as described in further detail below, the resistance of the strain gauge 166 changes when the cantilever portion 140 is flexed, thus changing the voltage of an analog signal representative of the uterine activity causing the cantilever portion 140 to flex.

As shown, the circuit board 142 includes a signal converter 168, a controller 170 (e.g., a microcontroller or microprocessor), and communication circuitry 172. The signal converter 168 may function as an analog-to-digital (A/D) converter and is configured to receive an analog signal from the sensor 164 corresponding to the strain on the cantilever portion 140. The signal converter 168 may then convert the analog signal to a digital signal for processing by the controller 170. In some forms, the signal converter 168 may be an HX711 load cell IC that is used to interface the strain gauge 166 and the controller 170.

The controller 170 is configured to process the signal received from the signal converter 168 and cause the communication circuitry 172 to communicate a corresponding signal to a remote computing device 174 or mobile communication device 176 (shown in FIG. 12). In some forms, the communication circuitry 172 is configured to communicate and relay a digital signal representative of the sensed strain to one or both of the remote computing device 174 and the mobile communication device 176 for additional processing. The communication circuitry 172 may in some forms utilize a short-range communication protocol such as, for example, Zigbee, Z-Wave, Bluetooth (or Bluetooth Low Energy (BLE)), Near Field Communications (NFC), Long Terminal Repeat (LTR), Wi-Fi, WiMAX, or other proprietary or public wireless communication methods. By one approach, the communication circuitry 172 includes a universal asynchronous receiver/transmitter (UART).

As described in further detail below, such a digital signal may be communicated to a remote computing device 174 or mobile communication device (e.g., a device associated with a healthcare professional or other third party) to provide information regarding the status of the user's pregnancy or to provide an alert in case of emergency or other time sensitive events.

In some embodiments, the circuit board 142 may include a global positioning system (GPS) chip (not shown) to provide GPS data indicative of a location of the device 100. Additionally or alternatively, the communication circuitry 172 may include a subscriber identity module (SIM) card such that the device 100 may be interfaced over a mobile telephony network. In some forms a SIM card is included on circuit board 142. The SIM card and controller 170 may be configured such that the location of the device 100 may be determined via the telephony network using, for example, triangulation of signals received from cell towers.

The power source 160 positioned in the inner cavity is electrically coupled to the circuit board 142 via wires 162 to provide power to the various components thereof (e.g., signal converter 168, controller 170, and communication circuitry 172). In some embodiments, the power source 160 is a rechargeable lithium ion battery, which may be electrically coupled to the charging port 134 such that the device 100 does not require replacement of the power source 160. In other forms, the device 100 may be configured to receive replaceable batteries, for example, AA or AAA batteries. Additionally, the power source 160 may be coupled to one of the LEDs 132 visible on the upper portion 114 of the housing 102 to indicate whether the power source 160 is running low on power, and a recharge or replacement is required.

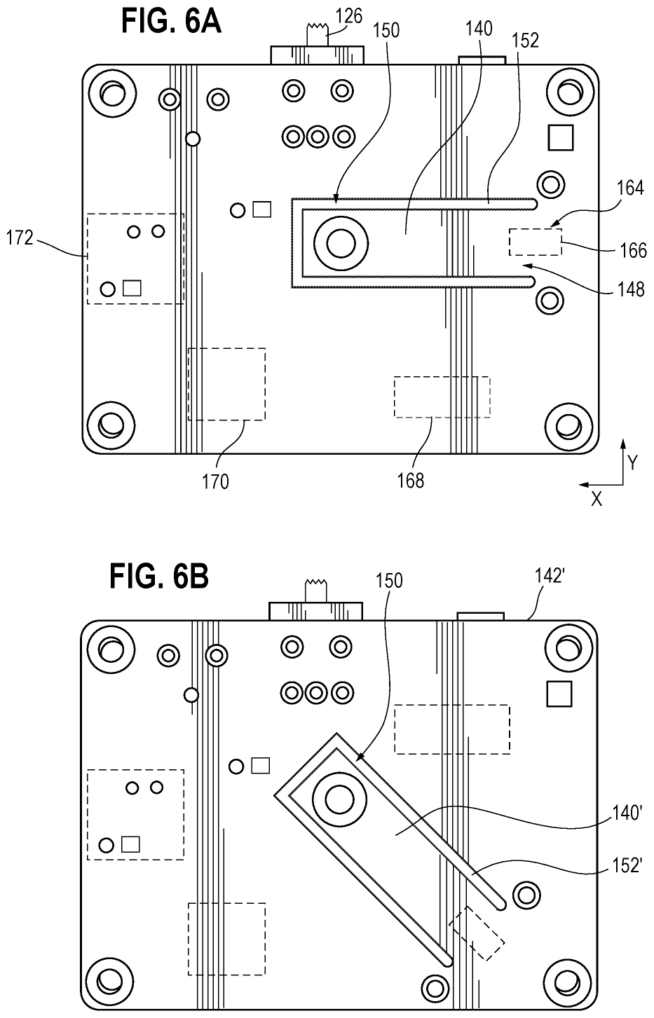

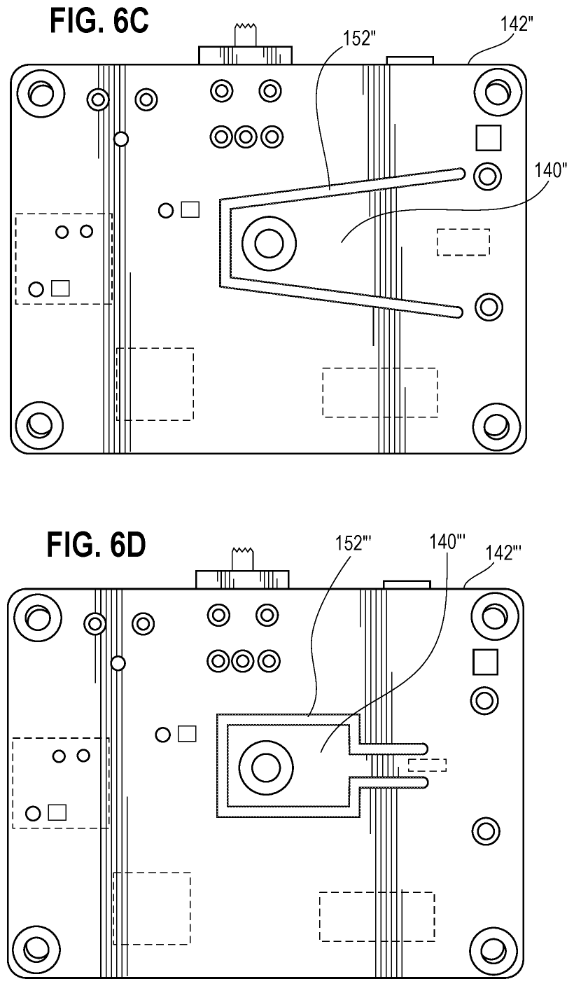

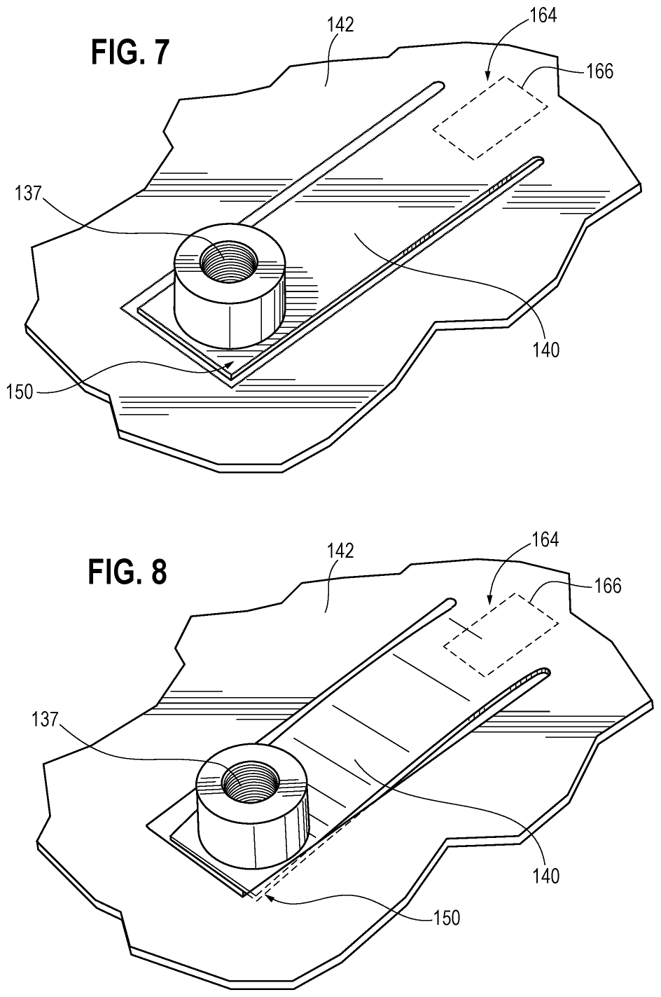

Referring to FIG. 6A, the circuit board 142 may be a printed circuit board (PCB) including various electrical components. In some forms, the material properties of the circuit board 142 may affect the flexing characteristics of the cantilever portion 140. For example, the thickness of the circuit board 142, the type of glass mat utilized, the lamination of the layers (e.g., the “stack up”), whether copper layers are included, the direction of the fibers in the glass mat with respect to the slot 152, the positioning of the apertures 154 for anchoring to the housing 102 the portion of the circuit board 142 proximate the attached end 148 of cantilever portion 140, etc., may all affect the stiffness, linearity, elastic limit, and fatigue of the circuit board 142 and cantilever portion 140 thereof. In one embodiment, the circuit board 142 is formed of glass fibers woven in both direction X and direction Y as illustrated in FIG. 6A, and the at least one slot defining the cantilever portion is preferably formed parallel to the woven fibers such that the slot is generally aligned therewith. In one embodiment, the circuit board 142 may be formed of FR4 epoxy glass and have a thickness of about 1.6 mm. The circuit board material and properties may be selected to provide the desired stiffness and other properties mentioned above of the cantilever portion 140 such that it may bend or flex about attached end 148. In addition to the properties of the circuit board material and the orientation of the cantilever portion 140 with respect to the weave of the fibers in the circuit board, the size and shape of the cantilevered portion 140 may also be selected to obtain desired strain measurements and/or sensitivity of the sensor 164 to uterine activity.

Referring to FIG. 6B, an alternative circuit board 142′ is shown having an alternate arrangement of a cantilever portion 140′. As illustrated, the cantilever portion 140′ is positioned at an angle with respect to the weave of the circuit board 142′ in contrast with the cantilever portion 140 in FIG. 6A shown extending generally parallel to the weave of the circuit board 142 in directions X and Y. In some embodiments, slot patterns formed in the circuit board 142′ to define the cantilever portion 140′ can be selected to provide the desired stiffness or flexing properties thereof. The free end 150′ is sized and shaped to permit coupling to the actuator 136.

In some forms, the at least one slot 152′ may be cut in the circuit board 142′ to form a cantilever portion having the desired size, shape, and width such that various characteristics thereof (e.g., stiffness and Young's Modulus) may be achieved. For example, as shown in FIG. 6C, the cantilever portion 140″ of circuit board 142″ may be defined by slot 152″ to form a generally trapezoidal shape. FIG. 6D illustrates still another form of the cantilever portion 140′″ in which the cantilevered portion 140′″ has a paddle-like shape with a smaller width proximate the attached end thereof and a greater width proximate the free end. Other shapes, sizes, and orientations are within the spirit and scope of the present invention.

FIGS. 7 and 8 illustrate a cut-away view of the cantilever portion 140 of the circuit board 142 showing an at-rest state (FIG. 7) of the free end 150 of the cantilever portion 140 and a flexed state (FIG. 8) in which the free end 150 has been pushed downward by the actuator 136 (shown in FIGS. 2, 3, and 14). The actuator 136 and fastener 144 coupling the actuator 136 to a bore 137 have been omitted from FIGS. 7 and 8 for clarity and ease of illustration. As shown, the sensor 164 in the form of a strain gauge 166 is positioned on the side 158 of the circuit board 142 opposite the side from which actuator 136 protrudes. Sensor 164 is positioned proximate the attached end 148 of the cantilever portion 140. As the free end 150 is flexed between the states shown in FIGS. 7 and 8, the resistance of the strain gauge 166 changes due to the physical stretching of a conductor positioned therein. An analog signal representative of the voltage or current resulting from this change in resistance is received by the signal converter 168.

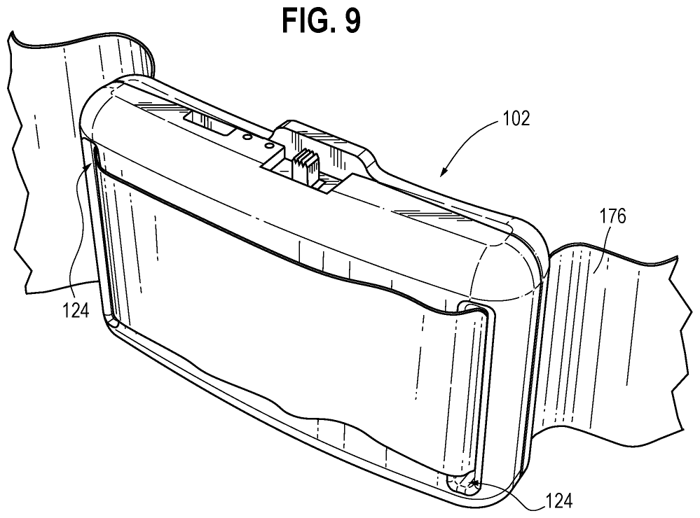

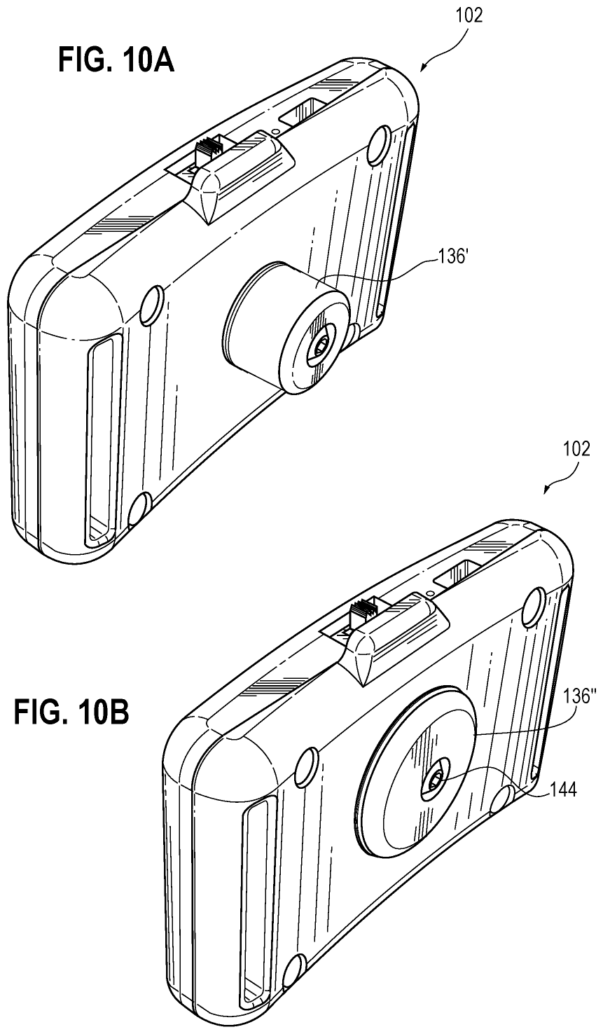

As shown in FIG. 9, a belt 176 may be coupled to the device 100 via the slots 124 to secure the device 100 (and actuator 136) against the abdomen of the user. The belt 176 may be formed of an elastic or stretchable material such that the device 100 will be held with the actuator 136 securely pressed against the abdomen without unduly restricting blood flow in the area. Additionally, the belt 176 may optionally include a liner on an interior face thereof to minimize or inhibit transmission of radiation to the abdominal area of the user. In other embodiments, the housing 102 of the device 100 may not include the openings 122 and the device 100 may, for example, alternatively be secured to the abdomen of the user via an adhesive or be integrated into clothing such as a shirt of the user.

As described above, the actuator 136 may be formed having different sizes and shapes to accommodate users having a range of body types and body mass indexes (BMIs). Various alternative embodiments of the actuator may be interchangeable such that the user may select an actuator suitable for their comfort level and body type (e.g., their individual BMI) and attach it to the device 100 before the device 100 is secured to the abdomen. Examples of alternative forms are illustrated in FIGS. 10A and 10B. Referring to FIG. 10A, an alternative actuator 136′ is shown having an annular shape similar to actuator 136, but having an increased axial length to protrude farther out from the front side 106 of housing 102 and a smaller surface area relative thereto, which may be suitable, for example, for users having a higher than average BMI. In FIG. 10B, another alternative actuator 136″ is shown having a shorter axial length relative to other actuators 136, 136′ and a larger surface area for contacting the abdomen of users, for example, those users having a lower than average BMI. Thus, the actuator may have various shapes and sizes. Regardless of shape and size, the actuator is coupled to the free end 150 of the cantilever portion 140 such that uterine activity may be sensed.

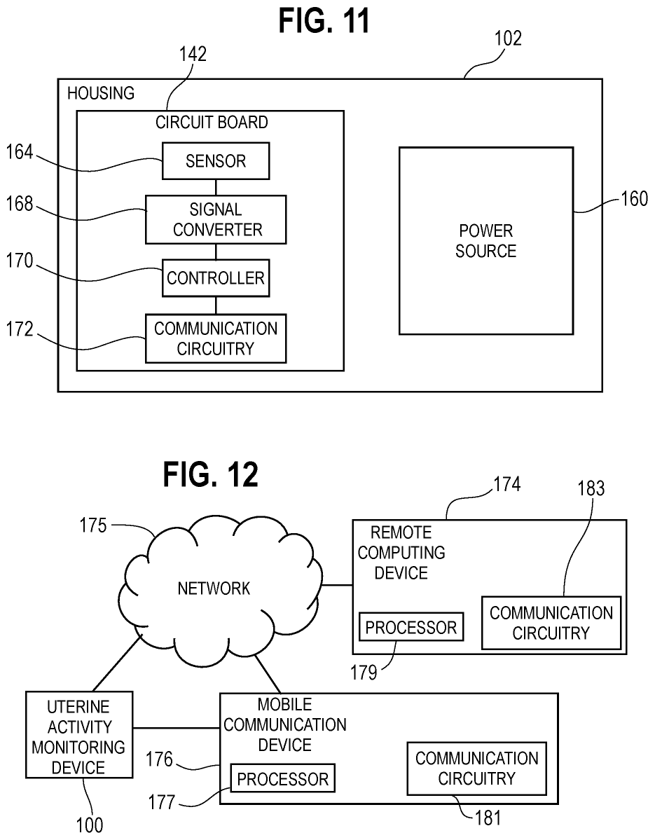

An example of the operation of the device 100 for monitoring uterine activity and assessing whether the user has entered the first stage of labor will now be described with respect to FIGS. 11 and 12. Specifically, FIG. 11 is a schematic diagram illustrating the main components of the device 100, and FIG. 12 is a schematic diagram illustrating an example of a communication scheme for the device 100. As described above, the device 100 is configured to be worn by a user such that at least the actuator 136 of the back side 106 of the housing 102 is in contact with the user's abdomen to detect uterine activity (e.g., a contraction). Upon occurrence of a contraction, the user's abdomen will move outward and inward, causing the actuator 136 to deflect the free end 150 of the cantilever portion 140 of the circuit board 142. The resistance of the strain gauge 166, which in one embodiment is proximate the attached end 148 of the cantilever portion 140, changes as the free end 150 is deflected by actuator 136, and an analog voltage or current signal that is a function of the resistance of strain gauge 166 is converted by the signal convertor 168 into a digital signal for the controller 170. The controller 170 may then cause the communication circuitry 172 (e.g., a Bluetooth® chip) to communicate a digital signal representative of the detected uterine activity to, for example, the remote computing device 174 (e.g., a cloud computing device) or to the mobile communication device 176, or both. The remote computing device 174 comprises a processor 179 and communication circuitry 183. In some embodiments, the remote computing device 174 may comprise or may be communicatively connected to a mobile communication device 176 of the user. For example, the mobile communication device 176 may comprise a smart phone, a tablet, or device of the user connected to the device 100 (e.g., via a network 175 such as the internet, or a short-range communication protocol) and may include a processor 177 and communication circuitry 181.

Once the digital signal representative of uterine activity of the user is received by either the remote computing device 174 or the mobile communication device 176, the signal may be further filtered, amplified, or conditioned for analysis and analyzed to determine whether, for example, a contraction occurred. During ordinary operation, the device 100 is configured to continuously monitor uterine activity such that multiple contractions may be detected over an interval of time, and this information processed to determine, for example, whether the user has entered the first stage of labor. So configured, such continuous monitoring may allow the detected contractions to be displayed to the user in real-time through an application on the remote computing device 174 or on the user's mobile communication device 176.

The processor 177 of the mobile communication device 176 or the processor 179 of the remote computing device 174 may be configured to analyze the signal representative of the monitored uterine activity to, for example, differentiate between first stage labor contractions, ordinary muscle contractions, or Braxton-Hicks contractions. The processor may be configured to perform such differentiation based at least in part on a comparison between the signal representative of the detected uterine activity and a specified threshold relating to the magnitude, the time interval between detected uterine activity signals, or both. For example, the threshold may be based on statistical averages for uterine activity of a specific age group or population, or alternatively may be specific to the patient. In some forms, the processor 179 of the remote computing device 174 or the processor 177 of the mobile communication device 176 may be configured to record the monitored activity in a memory coupled thereto and may establish a baseline of measured values of the user. Throughout the pregnancy of the user, the processor 177 and/or 179 may be configured to update and analyze changes in the user's baseline values via a machine learning algorithm, such that the accuracy of the detection and differentiation of contractions may be improved. For example, such processors may be configured to use decision trees, thresholding, Bayesian networks, Markov chains, or artificial neural networks to analyze the detected uterine activity and determine whether first stage labor has begun based, in part, on the magnitude of and time intervals measured between the detected contractions. Additionally or alternatively, some or all of the signal processing and determining functions may be performed via the controller 170 of the device 100.

In one embodiment, if the processor 179 of the remote computing device 174 or the processor 177 of the mobile communication device 176 receives signals indicating that contractions are occurring at less than four-minute intervals, for at least one minute each, lasting for at least one hour, the respective processor may determine that the user has entered labor. Such determinations may also incorporate various characteristics of the user, such as age, which may be input into a user interface associated with, for example, the remote computing device 174 or mobile communication device 176. For example, in some cases, the age of the pregnant user may affect the frequency or duration of the contractions typically present during the first stage of labor.

Upon the remote computing device 174 or mobile communication device determining that first stage labor has begun, either device 174 or 176 may be configured to communicate a notification to the user or another party via the communication circuitry 183 or 181, respectively. For example, the remote computing device 174 may be configured to cause communication of a notification signal to the mobile communication device 176 when uterine activity significantly changes relative to the threshold or falls outside of a preset range. In some forms, the mobile communication device 176 may be used by the user, a medical services provider, or a family member of the user. In response to receiving the notification signal, the mobile communication device 176 may display a message indicating that the user should seek medical attention because labor is imminent. Additionally or alternatively, the notification signal may be configured to display a graph, message, picture, etc. corresponding to detected uterine activity. In still other forms, the notification signal may be further communicated to mobile communication devices of a third-party such as a family member or healthcare professional. In embodiments where the device 100 includes a GPS chip or other circuitry for determining location (e.g., a subscriber identification module (SIM card)), the remote computing device 174 may be further configured to communicate a notification to any number of third-party devices indicating the physical location of the user upon detection that the user has entered the first stage of labor.

As briefly described above, various embodiments of the device 100 provided herein may be used in connection with an application on the mobile communication device 176. For example, the device 100 may be paired to the mobile communication device 176 via Bluetooth® or other short-range communication protocols to provide real-time information to the user. In one embodiment, the user is able to access the application to view a graphical representation of real-time uterine activity measured by the device 100 as the cantilever portion 140 is flexed by the actuator 136 over time. Such an application may permit the user to share the real-time viewing of the detected uterine activity (e.g., contractions) with family and friends, or a healthcare provider. In some forms, the application may be connected to a web-based application for viewing online.

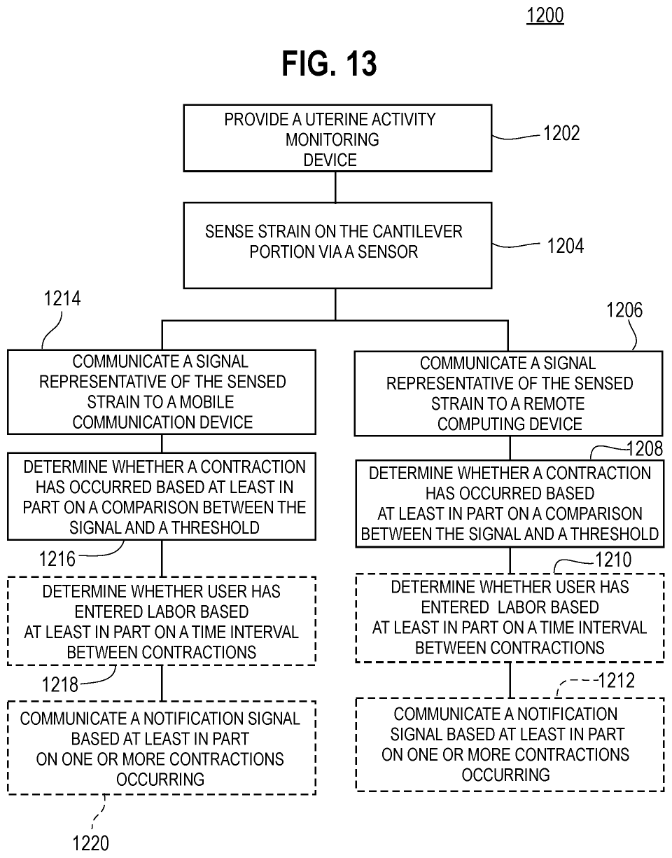

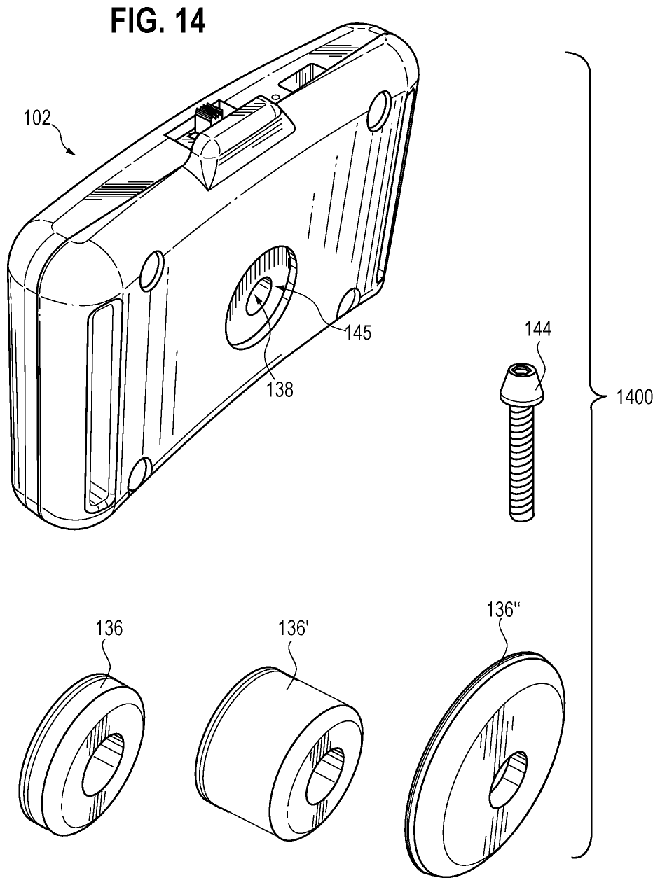

Referring now to FIG. 13, a method 1200 is shown for detecting uterine activity of a user. The method 1200 includes the steps of providing a uterine activity monitor device as described above at step 1202, the actuator of which may be positioned proximate the abdomen of the user. At step 1204, the device is configured to sense via a sensor such as a strain gauge strain on the cantilever portion resulting from uterine activity. The device may be configured to communicate a signal representative of the sensed strain and uterine activity to either a remote computing device, a mobile communication device, or both.

In some embodiments, the method 1200 may include the step 1206 of communicating a signal representative of the sensed strain to the remote computing device, and step 1208 of determining whether a contraction has occurred based at least in part on a comparison between the signal and a threshold. Optionally, at step 1210, the remote computing device may be configured to determine whether a user has entered labor based at least in part on a time interval between detected contractions. In addition, at step 1212, the remote computing device may be configured to communicate a notification signal based at least in part on the determination of whether the user has entered first stage labor.

In other forms, the method 1200 may additionally or alternatively include the step 1214 of communicating a signal representative of the sensed strain to the mobile communication device, and step 1216 of determining whether a contraction has occurred based at least in part on a comparison between the signal and a threshold. Optionally, at step 1218, the mobile communication device may be configured to determine whether a user has entered labor based at least in part on a time interval between detected contractions. In addition, at step 1220, the mobile communication device may be configured to communicate a notification signal based at least in part on the determination of whether the user has entered first stage labor.

Referring now to FIG. 14, a kit 1400 is provided including the uterine activity monitoring device with the fastener 144 removed and the actuator 136 detached, and further including one or more alternative actuators, such as actuators 136′, 136″, for accommodating users having different body types and BMIs. As illustrated, the back side 106 of the housing 102 includes a recessed portion 145 such that the actuators 136, 136′, 136″ may be partially received therein when impinged upon by the user. So configured, the recessed portion 145 permits the actuator to be seated at least partially therein when the cantilever portion 140 is in a flexed configuration (e.g., as shown in FIG. 8). Optionally, the kit 1400 may include an instruction sheet or data sheet including a suggested actuator (e.g., 136, 136′, 136″) for different body types and ranges of BMIs. For example, the user may obtain the kit 1400 including the different actuators 136, 136′, 136″ and select and attach an actuator using the fastener 144 through the aperture 138 in the housing 102. So configured, the actuators in the kit 1400 may be interchangeable for use with the device. In some forms, the kit 1400 may include various types of fasteners as described above (e.g., a hex bolt or other fastener), and may also include a driver (e.g., a screwdriver or hex driver) corresponding with the fastener for attaching the selected actuator to the device.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A uterine activity sensor device comprising:
a housing including an inner cavity having a circuit board disposed therein, the circuit board including circuitry and a sensor;
wherein the circuit board further comprises at least one slot formed therein that defines a cantilever portion of the circuit board, the cantilever portion having an attached end and a free end; and
wherein the cantilever portion is configured to flex upon movement of an actuator coupled to the free end thereof, and wherein the sensor is positioned proximate the attached end of the cantilever portion and is configured to sense strain on the cantilever portion.

2. The device of claim 1, wherein the sensor comprises a strain gauge.

3. The device of claim 2, wherein the strain gauge comprises at least one of a foil gauge, a silicon gauge, and a polymer on the circuit board.

4. The device of claim 2, wherein the circuitry comprises a signal converter, a controller, and communication circuitry, and wherein the controller is configured to cause the communication circuitry to communicate a signal representative of the sensed strain to a remote computing device.

5. The device of claim 4, wherein the circuitry further comprises a global positioning satellite (GPS) chip.

6. The device of claim 4 wherein the circuitry further comprises a subscriber identification module.

7. The device of claim 4, wherein the remote computing device is configured to determine whether a contraction has occurred based at least in part on a comparison between the signal representative of the sensed strain and a threshold.

8. The device of claim 4 wherein the remote computing device comprises a mobile communication device.

9. The device of claim 4, wherein the communication circuitry is configured to communicate via a short-range communication protocol comprising at least one of Zigbee, Z-Wave, Bluetooth, Bluetooth Low Energy, Near Field Communication, Long Terminal Repeat, Wi-Fi, and WiMAX.

10. The device of claim 2, wherein the circuitry comprises a signal converter, a controller, and communication circuitry, and wherein the controller is configured to cause the communication circuitry to communicate to a mobile communication device a signal representative of strain sensed by the strain gauge.

11. The device of claim 1, wherein the housing further comprises an aperture extending through a portion thereof, and wherein the actuator extends at least partially through the aperture.

12. The device of claim 1, wherein the circuit board comprises at least one aperture extending therethrough, the at least one aperture positioned proximate the attached end of the cantilever portion and configured to receive a fastener therethrough to couple the circuit board to the housing.

13. The device of claim 1, wherein at least one location on the circuit board proximate the attached end of the cantilever portion is anchored to the housing.

14. The device of claim 1, wherein a side of the at least one slot is generally aligned with a weave of fibers forming the circuit board.

15. The device of claim 1, wherein the at least one slot is formed at an angle relative to the weave of fibers forming the circuit board.

16. The device of claim 1, wherein the cantilever portion is of a generally rectangular shape.

17. The device of claim 1, wherein the housing has an arcuate configuration and a portion of the actuator protrudes from a concave side thereof.

18. The device of claim 1, wherein the housing comprises at least one opening therethrough for receiving at least a portion of a strap for securing the housing proximate a user's abdomen.

19. The device of claim 1, wherein the housing is formed having a width of about 3 inches, a height of about 2 inches, and a thickness of about 0.7 inches.

20. The device of claim 1, wherein a portion of the actuator is interchangeable with at least one other portion of an actuator having a different size than the portion.

21. The device of claim 1, wherein a portion of the actuator is interchangeable with at least one other portion of an actuator having a different thickness than the portion.

22. The device of claim 1, wherein a portion of the actuator is interchangeable with at least one other portion of an actuator having a different shape than the portion.

23. The device of claim 1, wherein the housing includes a recessed portion, and wherein the actuator may be received at least partially therein upon movement thereof.

24. A method of detecting uterine activity comprising:
providing the uterine activity sensor device of claim 1;
sensing, via the sensor, strain on the cantilever portion; and
communicating a signal representative of the sensed strain to a computing device.

25. The method of claim 24, wherein the computing device is a mobile communication device.

26. The method of claim 24, wherein the computing device is a remote computing device.

27. The method of claim 24, further comprising:
determining, at the computing device, at the remote computing device, whether a contraction has occurred based at least in part on a comparison between the signal and a threshold.

28. The method of claim 24, further comprising determining, at the computing device, whether a user has entered labor based at least in part on a time interval between contractions.

29. The method of claim 28, further comprising communicating a notification signal to a mobile communication device upon a determination that the user has entered labor.

30. A kit comprising:
the uterine activity sensor device of claim 1;
a fastener for coupling at least an end portion of the actuator to the free end of the cantilever portion; and
a plurality of interchangeable end portions of the actuator, each end portion being configured to facilitate detection of uterine activity of a user having a given body mass index (BMI).

31. The kit of claim 30 wherein the fastener is threaded, and wherein the kit further comprises a tool adapted for use with the threaded fastener.
